(19) United States
(12) Patent Application Publication
(10) Pub. No.: US 2015/0198581 A1
(43) Pub. Date: Jul. 16, 2015

(54) METHOD, MOBILE APPLICATION, AND SYSTEM FOR PROVIDING FOOD SAFETY MAP

(71) Applicant: National Tsing Hua University, Hsin Chu City (TW)

(72) Inventors: Ting-Lan WANG, Kaohsiung City (TW); Chen-Meng KUAN, Hengshan Township (TW); Po-Hsien CHIU, New Taipei City (TW); Jung-Chun KAO, New Taipei City (TW); Chao-Min Cheng, New Taipei City (TW)

(21) Appl. No.: 14/307,256

(22) Filed: Jun. 17, 2014

(30) Foreign Application Priority Data
Jan. 10, 2014 (TW) 103100952

(51) Int. Cl. G01N 33/487; C12Q 1/25; C12Q 1/54; G06T 11/00; G01N 33/66; G01N 33/92; G06T 7/00; G01N 21/78; G01N 33/84

(52) U.S. Cl. CPC G01N 33/48792; G01N 21/78; C12Q 1/25; C12Q 1/54; G01N 33/84; G01N 33/66; G01N 33/92; G06T 7/0002; G06T 11/001; G01N 2201/12; G01N 2458/00; G06T 2207/10004; G06T 2207/30128

(57) ABSTRACT

A method for providing food safety map comprises: immersing a reagent block of a food safety testing sheet into a specimen; using camera of a mobile device to take a picture of the reagent block to obtain a reactive image; analyzing the reactive image to obtain a food safety information; obtaining an address information according to the location; transmitting the food safety information and the address information to a server through a wireless connection; using that server to map the food safety information to a map according to the address information; and publishing the map to the Internet.

### FIELD OF THE INVENTION

The invention is relevant to food safety applications of the Internet, specifically, methods, mobile applications, and systems for providing food safety information.

### DESCRIPTION OF THE PRIOR ART

Detection reagents and testing papers are widely used to ensure food safety. However, no inexpensive and convenient detection tools are available for use by the public. Since no inexpensive and convenient detection tools are available for public use, the public must rely on government testing.

Since conventional detection test strips require special equipment or pumps to coat chemical reagents, the cost of production is very high. In the conventional test, reagents are directly immersed into the liquid specimen, and direct immersion dissolves the reagents, reduces the accuracy of the test, and changes the composition of the liquid of specimen. Since accuracy is easily disturbed by the external environment, achieving consistent qualitative analysis and semi-quantitative analysis results is difficult.

When the conventional test strip is used to do the test, the testing result cannot be obtained by observing the color change of the reagent block visually, and thus additional reading apparatus is required.

Using the conventional test strip is inconvenient and time consuming. Another disadvantage is the high cost of their long-term use since the test strips are consumables. Therefore, an inexpensive test strip is needed.

Finally, a publically available platform for integrating food safety information is needed.

### SUMMARY OF THE INVENTION

The invention aims to solve the above problems.

The invention uses a food safety testing sheet that avoids the above problems by enabling a quick inspection that substantially reduces test turnaround time. The inspection method is also inexpensive and can be performed at any time and place to ensure the food is safe.

This food safety testing sheet of the invention contains xylem fibers with micropores and capillary structures that absorb both the reagent and the liquid used to inspect for pesticide residues in tea and nitrites in food. Inexpensive materials and equipment and visual results make this sheet affordable, convenient, and effective.

This sheet has advantages compared conventional devices: (1) equipment or pumps are not required to coat the reagent on the food safety testing sheet of the invention during manufacturing, which reduces its cost; (2) Because the reagent block of the food safety testing sheet of the invention is not immersed into the liquid, the reagent is prevented from being dissolved into and contaminating the liquid and accuracy is ensured; (3) The food safety testing sheet of the invention allows the liquid to flow in the inner path, unaffected by environment, which improves qualitative analysis or semi-quantitative analysis accuracy; (4) The testing result is obtained by observing the color change of the reagent block visually, no additional reading apparatus is required; and (5) The xylem-fiber material is inexpensive, and reduces the production cost of the food safety testing sheet of the invention.

The invention provides a testing platform, which has the advantages of test paper without its disadvantages.

The invention provides a food safety information platform with a special food safety testing sheet. Users may test food of restaurants by the food safety testing sheets, and the test results can be uploaded into the cloud by the network. When many test results from restaurants are sent to the database in the cloud, the test results are combined with the geographic map to construct the food safety map.

When many test results from restaurants are sent to the database in the cloud, the test results are combined with the geographic map to produce the food safety map, where users can check the food safety information of restaurants and users can choose restaurants with better food safety records. Therefore, the invention motivates restaurants to improve their food safety.

Since mobile devices are now widely used, the invention uses mobile devices with internal cameras, e.g., smart phones and tablets, to enable real-time inspection anytime and anywhere. Meanwhile, the invention uses the mobile application to analyze and send data to the database of the cloud to construct a food safety map, and thus the scattering food safety information can be integrated and published to be read by users who want to find safe restaurants.

### BRIEF DESCRIPTION OF THE DRAWINGS

The primitive objectives and advantages of the invention will become apparent upon reading the following description and upon reference to the accompanying drawings in which:

FIG. 1A shows the front view of the food safety testing sheet of the invention;

FIG. 1B shows the side view of the food safety testing sheet in FIG. 1A;

FIG. 2A is a flowchart for providing the food safety map according to one embodiment of the invention;

FIG. 2B is a flowchart illustrating the details of the color correction processes shown in FIG. 2A according to one embodiment of the invention;

FIG. 3 is a flowchart for providing the food safety map according another embodiment of the invention;

FIGS. 4A-4C show the toothpicks adsorption results of different concentrations of glucose in the buffer system;

FIGS. 5A-5C show the toothpicks absorption results for different concentrations of cholesterol in the buffer system;

FIG. 6 shows reactive images of the toothpicks after exposure to varying concentrations of nitrite solution;

FIG. 7 is a photograph illustrating the testing results of stirring rods without and with surface treatments;

FIG. 8 shows two photographs displaying reactive images of stirring rods exposed to nitrite solution of different concentrations;

FIG. 9A shows the color intensities of the reagent blocks denoted by nitrite concentrations; and

FIG. 9B shows a chart based on the results in FIG. 9A. The relationship between color intensity and nitrite concentration is linear and has good compliance.

### DESCRIPTION OF THE PREFERRED EMBODIMENT

In order to fully understand the manner in which the above-recited details and other advantages and objects according to the invention are obtained, a more detailed description of the invention will be rendered by reference to the best-contemplated mode and specific embodiments thereof. The following description of the invention is made for the purpose of illustrating the general principles of the invention and should not be taken in a limiting sense; it is intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein. The terminology used in the description presented below is intended to be interpreted in its broadest reasonable manner, even though it is being used in conjunction with a detailed description of certain specific embodiments of the invention. Certain terms may even be emphasized below; however, any terminology intended to be interpreted in any restricted manner will be overtly and specifically defined as such in this detailed description section. Where the context permits, singular or plural terms may also include the plural or singular terms, respectively. Moreover, unless the word “or” is expressly limited to mean only a single item exclusive from the other items in a list of two or more items, then the use of “or” in such a list is to be interpreted as including (a) any single item in the list, (b) all of the items in the list, or (c) any combination of items in the list.

Preferred embodiments and aspects of the invention will be described to explain the scope, structures and procedures of the invention. In addition to the preferred embodiments of the specification, the invention is widely applicable in other embodiments.

The invention relates to a lignocellulosic detection device provided with a lignocellulosic substrate and a detection reagent as the main components. Lignocellulose, which is also known as lignified xylem tissue or wood, refers to a collective plant tissue formed with vascular cambium inward development. Additionally, lignocellulose is usually extracted from the lignified xylem tissue in trees and shrubs. Since the lignocellulosic substrate has a robust mechanical structure and relatively good resistance to acids or alkaline, its advantage over filter papers is its improved structural strength.

In principle, the shape and/or size of the lignocellulosic substrate is not limited and may vary according to the design requirements. Some common shapes of the lignocellulosic substrate may include but are not limited to cylindrical, rectangular, plate-like shapes.

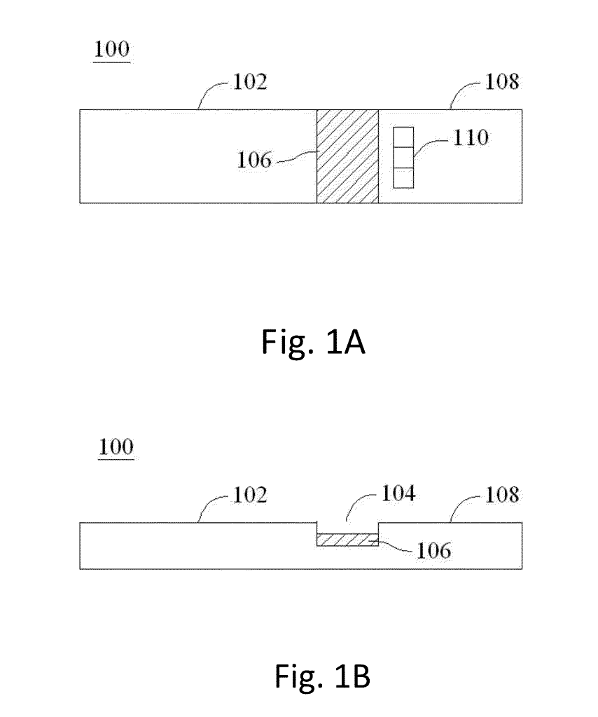

FIG. 1A shows the front view of the food safety testing sheet of the invention. FIG. 1B shows the side view of the food safety testing sheet in FIG. 1A. The food safety testing sheet (100) of the invention can be composed of lignocellulosic materials, which are inexpensive. FIGS. 1A and 1B show that the food safety testing sheet (100) of the invention includes an immersed end (102), which is immersed into the test specimen; a reagent block (104), which may be a groove coated by the reagent (106), and a handle end (108), which is held by the user to prevent contamination of the specimen and to keep the hands clean.

Generally, the shapes and sizes of the reagent block (104) can be varied on the basis of the requirements of the testing. The shape of the reagent block (104) in the embodiment of FIG. 1A is only illustrative and not thus limited. Generally speaking, the shape of the reagent block (104) may include without limitations to cuboid, cube, cylinder, hemisphere, V shape, other shapes, or combinations thereof. The position of the reagent block (104) may vary in different configurations required for detection.

In one embodiment, the reagent block (104) may be set as the detection zone, and the reagent (106) is provided in the reagent block (104) to carry out the detection. Therefore, no special equipment or pumps are needed to coat the reagent on the lignocellulosic sheet during manufacturing. In FIG. 1B, the reagent (106) is set at the bottom of the reagent block (104). However, the reagent (106) can also be set to single side, the bottom, bilateral sides of the groove or other combinations.

The internal transport channels (not shown) in the lignocellulosic material of the food safety testing sheet (100) are used to transport the specimen to be detected to the reagent block (104) via capillary action. Since the reagent block is not immersed in the liquid, the reagent does not dissolve into the liquid, which avoids contamination and ensures accuracy. Since the food safety testing sheet (100) also allows the liquid to flow in the inner path, it is unaffected by the environment, which improves the accuracy of qualitative or semi-quantitative analysis. A notable advantage is the avoidance of measurement errors caused by physical damage to the external transport channels of the food safety testing sheet (100) during the machining process. Another advantage is the controlled flow rate of the liquid specimen.

A surface treatment of the lignocellulosic substrate may also be performed to increase the stability of the lignocellulosic fiber. Surface treatments may include, but are not limited to, waterproofing treatment. Water proof reagents may include, but are not limited to PDMS (Polydimethylsiloxane). Alternatively, multiple assays include multiple qualitative and/or quantitative assays. For example, for increased detection diversity, multiple assays may include assays for detecting various substances or may include assays for detecting a single substance of varying concentrations.

In one embodiment, daily necessities can be used as the lignocellulosic substrate of the invention. Examples may include but are not limited to stirring rods, wooden chopsticks or toothpicks.

In one embodiment of the invention, toothpicks may be used as the lignocellulosic substrate. The toothpicks are commonly used to clean food stuck in the teeth gap as its general purpose and usually made of lignocellulosic materials including bamboo. Bamboo plants are shrubs or trees that have a lignocellulosic pole. Bamboo has a cost advantage due to the rapid growth of bamboo plants. Toothpicks have several advantages. Toothpicks are inexpensively and easily machined form natural materials. Toothpicks are readily available and are widely used for postprandial oral hygiene maintenance. One purpose of the invention is to develop a toothpick that can be used for both teeth cleaning and for disease detection. Therefore, no extra time is needed for disease detection.

The invention is principally directed at using the capillary action of the lignocellulosic substrate and the detection reagent adsorbed on the fibrous tissue of the lignocellulosic substrate to function as a disease detection platform. When a liquid specimen or a specimen containing liquid comes in contact with one end of the lignocellulosic substrate, the subject specimen is adsorbed to the lignocellulosic tissue of the lignocellulosic substrate by capillary action. The reaction with the detection reagent can then be used for specimen detection.

The detection reagent can be adsorbed into the lignocellulosic substrate by soaking the lignocellulosic substrate in the detection reagent solution. The capillary action of the lignocellulosic substrate and vascular tissues of the plant fiber cause adsorption of the detection reagent onto the lignocellulosic substrate. Here, a drying step may be performed for obtaining a lignocellulosic substrate for detection after the lignocellulosic substrate is soaked in the detection reagent solution.

The lignocellulosic substrate of the invention can use colorimetric reaction or fluorescence. Therefore, the detection reagent comprises a colorimetric agent or a fluorescent reagent. Colorimetric agents or fluorescent reagents used as detection reagents may be chosen based on the detection target. The change in the color of the reagent block indicates the test results. No additional reading apparatus is required.

In one embodiment of the invention, the detection reagent may comprise an enzyme. As mentioned above, qualitative measurements of substances and quantitative measurements of substance concentrations may be performed by measuring changes in color or other properties caused by the reaction between enzyme and substances in the solution. For example, color changes in the test strips used in the glucose oxidase assay result from specific reactions catalyzed by glucose oxidase and glucose.

Examples of specimens include but are not limited to saliva, blood, urine or other body fluids. As mentioned above, the specimen may be absorbed to the fibrous tissue of the lignocellulosic substrate by capillary action to react with the detection reagent to achieve specimen detection. Solid substances to be detected may also be suspended and dissolved in a liquid solvent and then detected by the above described detection method using capillary action.

Notably, the lignocellulosic substrates used in the invention are widely used in eating or drinking utensils. For example, since lignocellulosic substrates such as toothpicks are used in postprandial oral hygiene maintenance, a preferred embodiment refers to the saliva of the test subjects. Advantages of saliva testing include noninvasiveness, convenient acquisition, very low cost, low risk of infection, and the ability to test and collect samples independently of medical staff.

Continuing the above description, the lignocellulosic substrate used for detection in the invention may be used for testing biochemical properties, such as glucose, nitrite, pH, of saliva, blood, urine or other body fluids. The following examples are given only to illustrate the principle and applications of the invention for detection.

After reacting with digestive enzyme in the saliva, Nitrate ions of the food will become nitrosamines, which identified as carcinogenic by medical researchers. Additionally, large amounts of nitrites may cause direct poisoning after excessive uptake of nitrite from drinking water, vegetables, grain, fish, meat, salted products. Since nitrite test strips that use chemical colorimetric reaction are now commerically available, food would be a suitable target for nitrite detection.

By applying the principle described above for measuring blood sugar or urine glucose, patients can use the device for self-monitoring of glucose at any time.

The pH of a solution is usually measured with litmus paper. Litmus paper is produced by immersing paper in a solution containing litmus reagent. The litmus reagent appears red in an acidic solution and blue in an alkaline solution. A lignocellulosic substrate prepared as described above can be used to detect pH.

The invention is further illustrated by the following working examples, which should not be construed as further limiting.

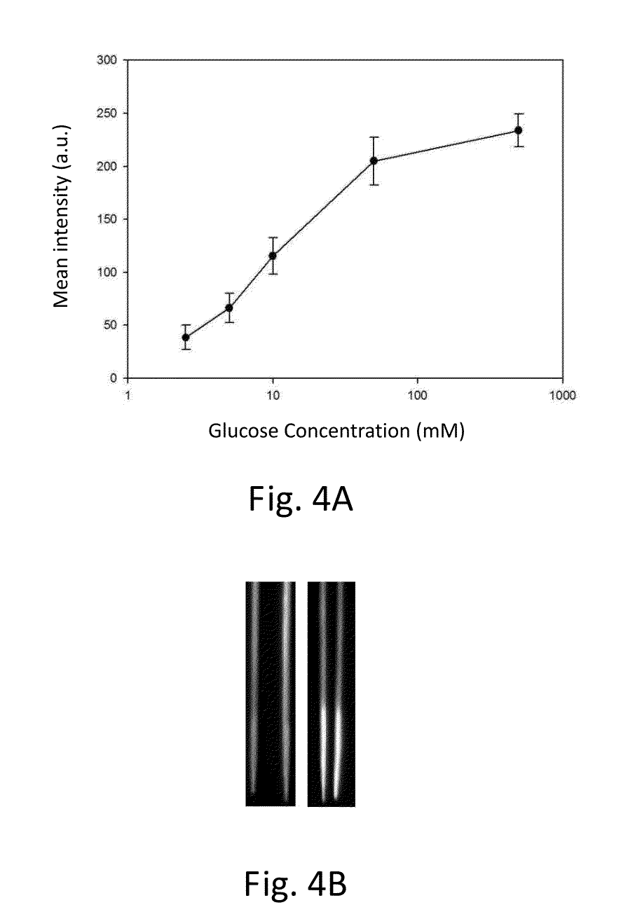

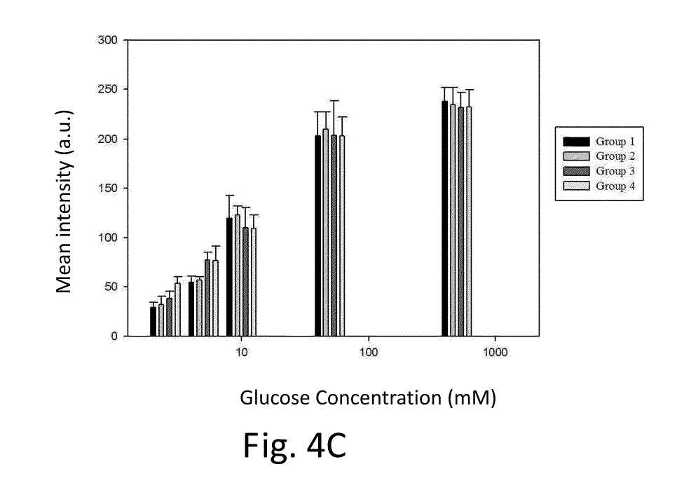

Preparation and assay methods of using a toothpick for measuring glucose are as follows: (1) soak the toothpick in 330 ul glucose detection solution containing Glucose oxidase 75 units/mL, of HRP 15 units/mL, fluorescent dye 10-acetyl-3,7-dihydroxyphenoxazine 300 μM (all drugs are available from Sigma-Aldrich); (2) soak the toothpick for five minutes; (3) remove the toothpick, and allow it to dry for 2 hours; and (4) use the dried toothpicks for glucose detection. FIGS. 4A-4C shows the toothpick adsorption results for varying concentrations of glucose in the buffer system. FIG. 4A is a calibration curve showing the corresponding relationship between average fluorescence intensity and glucose concentration (N=20). FIG. 4B is a photograph displaying fluorescence reactive image of toothpick immersed in the buffer system containing 2.5 mM and 500 mM glucose, respectively. FIG. 4B is a photograph showing fluorescence reactive images of toothpicks immersed in the buffer system containing 2.5 mM and 500 mM glucose, respectively. FIG. 4C is a diagram of the variations between different test groups with the same glucose concentration set (each group N=5). The average changes for glucose concentrations of 2.5, 5, 10, 50 and 500 mM were 0.633, 0.336, 0.118, 0.034 and 0.026, respectively.

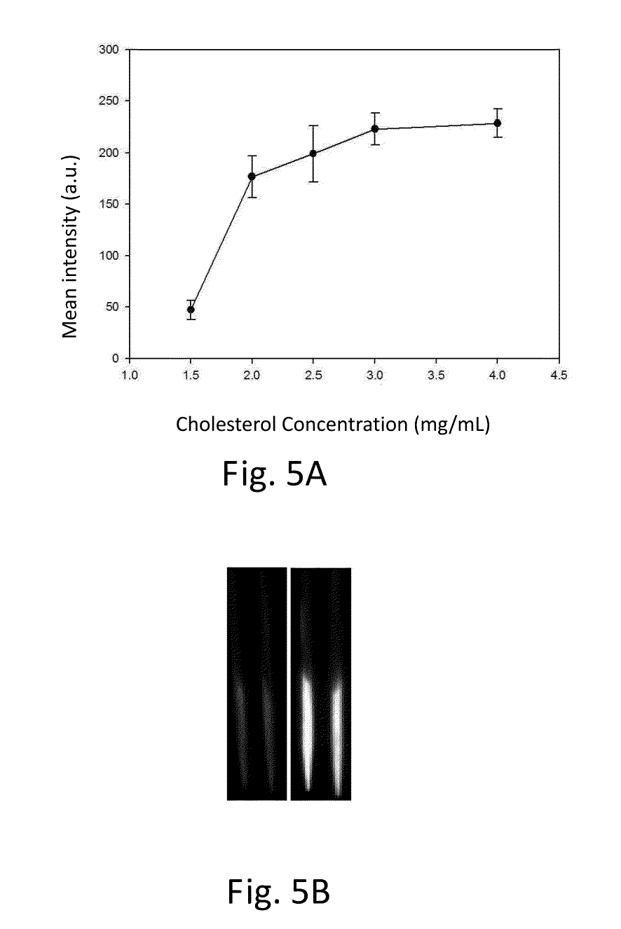

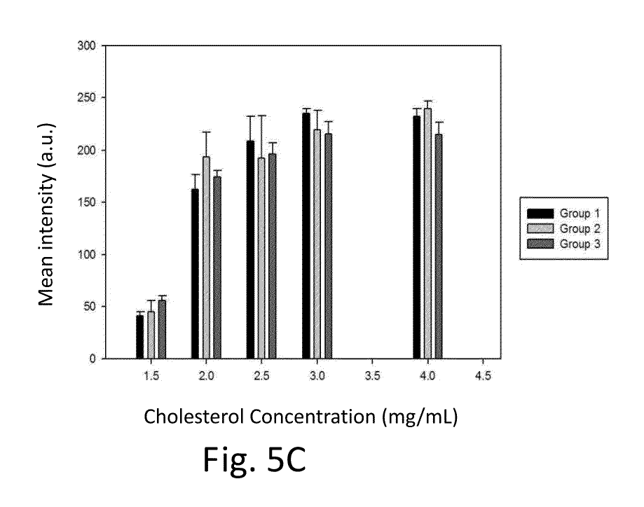

Preparation and assay methods for using toothpicks to measure cholesterol are as follows: (1) soak the toothpicks in the 330 μL cholesterol detection reagent containing cholesterol oxidase 2 units/mL, HRP 2 units/mL, fluorescent dye 10-acetyl-3,7-dihydroxyphenoxazine 300 μM (all available from Sigma-Aldrich); (2) soak the toothpick for five minutes; (3) remove the toothpick and allow to dry for 0.5 hours; (4) use the dried toothpick for cholesterol detection. FIGS. 5A-5C show the toothpick absorption results for varying cholesterol concentrations in the buffer system. The calibration curve in FIG. 5A shows the corresponding relationship between average fluorescence intensity and cholesterol concentration (N=15). FIG. 5B is a fluorescence reactive image of a toothpick immersed in the buffer system containing 1.5 mg/mL and 4 mg/mL cholesterol. FIG. 5C is a diagram illustrating the variations between different test groups with the same glucose concentration set (each group n=5). The average changes for glucose concentrations of 1.5, 2, 2.5, 3 and 4 mg/mL were 0.306, 0.177, 0.081, 0.088 and 0.107, respectively.

The toothpicks used in the nitrite detection device are prepared as follows. Toothpicks are coated with a nitrite detection reagent containing 50 mmol/L sulfanilamide (≧99%, Sigma-Aldrich), 330 mmol/L citric acid (≧99.5%, Sigma-Aldrich), and l0 mmol/L N-(1-naphthyl)ethylenediamine (≧98%, Sigma-Aldrich).

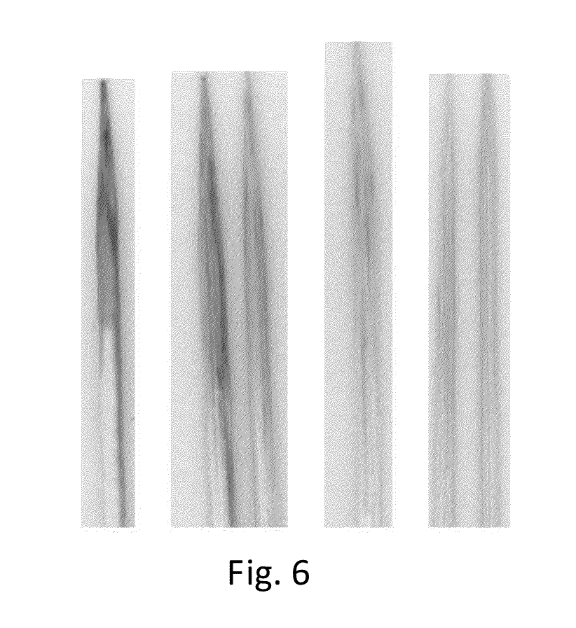

FIG. 6 shows reactive images of the toothpicks after exposure to varying concentrations of nitrite solution. The image shows, from left to right, the results for nitrite concentrations of 10, 5, 2.5, 1.25, 0.625 and 0.156 mM.

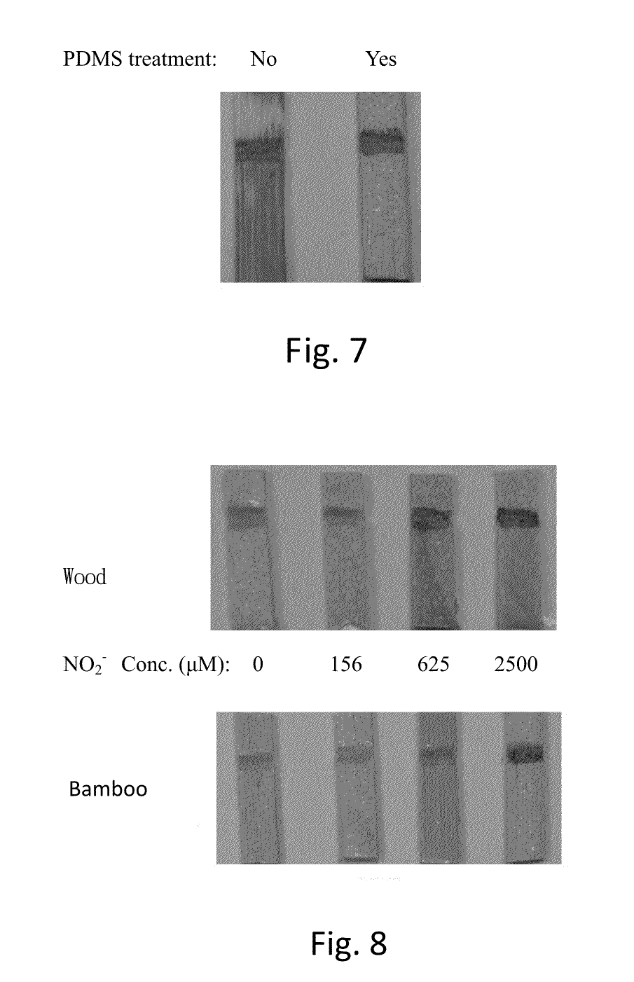

The surface of the stirring rod was waterproofed by applying a PDMS coating. One end of the stirring rod was then immersed in the red ink. FIG. 7 shows the results, which confirmed that the surfaces of the PDMS-coated stirring rods were water proof, i.e., the red ink was transported internally to the detection zone (104) in the groove. In the control group, the surface was covered with red ink, i.e, the red ink passed to the surface.

The stirring rods of the invention can be used to detect glucose. For example, wood and bamboo stirring rods were surface treated with, 4 μL PDMS. A 5 μL quantity of nitrite detection reagent was dropped within the detection zone (104). The nitrite detection reagent contained 50 mmol/L sulfanilamide, 330 mmol/L citric acid (≧99.5%, Sigma-Aldrich), 10 mmol/L N-(1-naphthyl)ethylenediamine. FIG. 6 is an illustration of the wood and bamboo stirring rods coated with the nitrite detection reagent and then exposed to nitrite solutions of varying concentrations. From left to right, the experimental results are shown for nitrite concentrations of 0, 156, 625 and 2500 μM.

In summary, the advantages of the lignocellulosic detection device in the invention include its fabrication from natural materials, its low cost, and its easy machining process. These advantages make it applicable for use in a disease diagnosis platform or substrate. The detection device in the invention can also diagnose disease more rapidly compared to conventional detection methods.

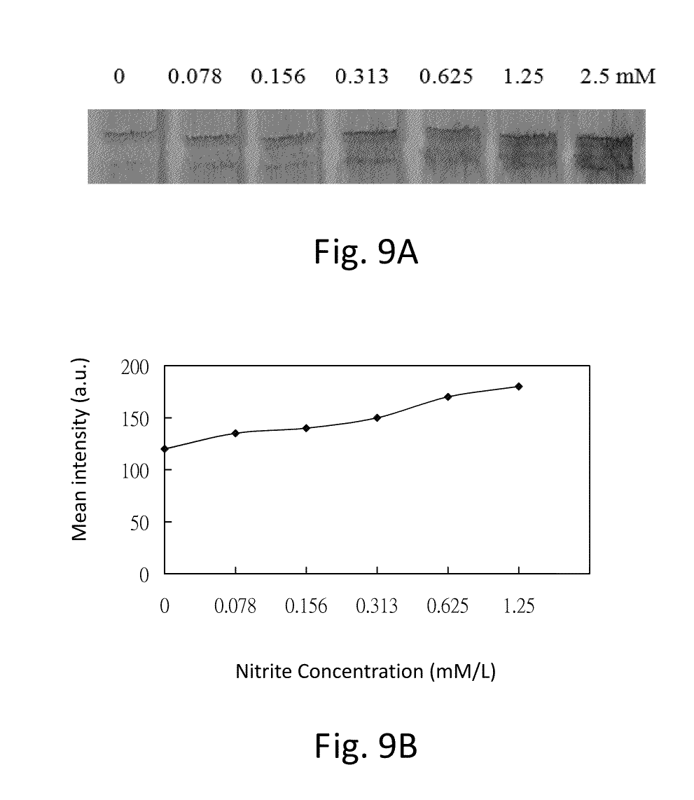

FIG. 9A shows the color intensities of the reagent blocks denoted by nitrite concentrations. FIG. 9B shows a chart based on the color intensity results in FIG. 9A. The color intensities and the nitrite concentrations show a consistent linear relationship. Since experiments have shown that nitrites are a key cause of cancer, the food safety testing sheet can be used to perform inspections with a short turnaround time. The inspection can be performed inexpensively and at any time and place to ensure the food is safe.

The food safety testing sheets can also be used to test restaurant food, and the test results can be uploaded into the cloud by the network. When test results for many different restaurants are sent to the cloud database, the test results are combined with the geographic map to construct the food safety map, where users can check the food safety information of restaurants to identify the restaurants with the best food safety records. Therefore, the test system motivates restaurants to improve their food safety.

Since mobile devices are widely used, the invention uses mobile devices with built-in cameras, such as smart phones and tablets, to enable real-time inspection at any time and place. Meanwhile, the invention uses a mobile application to analyze and send data to a cloud database. The data are then used to construct a food safety map so that various food safety information can be integrated and published to be read by users who want to find safe restaurants.

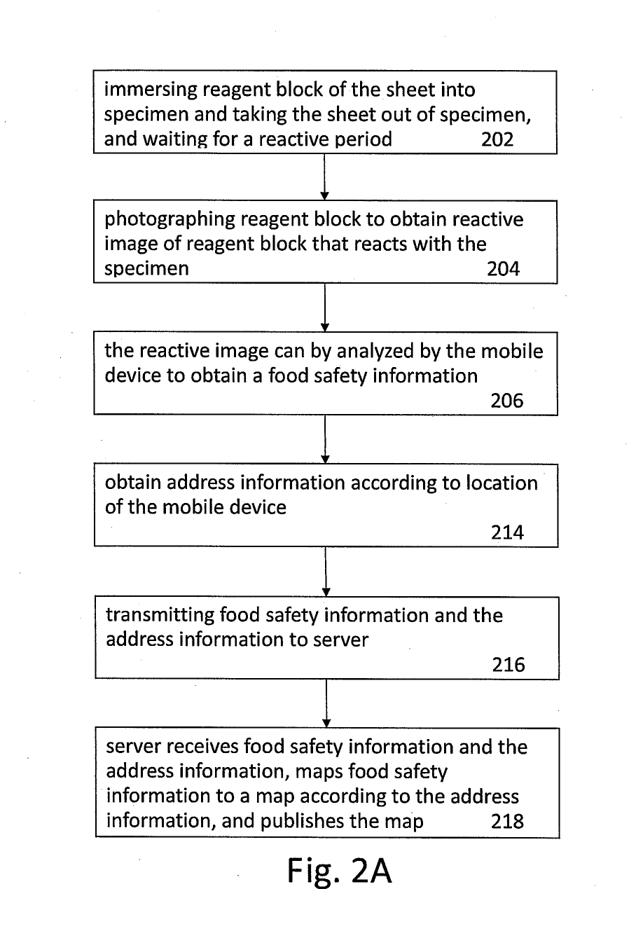

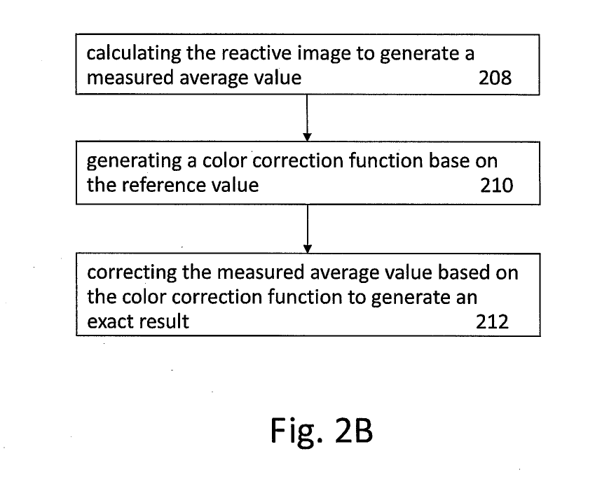

FIG. 2A is a flowchart of the procedure for creating a food safety map according an embodiment of the invention. FIGS. 1A and 1B illustrate a method creating a food safety map in the following steps: immerse a reagent block (104) of a food safety testing sheet (100) into a specimen; remove the food safety testing sheet (100) from the specimen; and wait for a reactive period after the food safety testing sheet (100) is removed from the specimen. The color of the reagent block (104) should change after coming in contact with the specimen (step 202).

A mobile device with a camera can be used to photograph the reagent block (104) to obtain a reactive image of the reagent block (104) that reacts with the specimen (step 204). Further, a trichromatic block (110) with red, green, and blue colors is set near the reagent block (104). When the reactive image of the reagent block (104) is obtained, the trichromatic block (110) mounted near the reagent block (104) is also used as a reference part of the reactive image. The reference part of the reactive image can be calculated to generate a measured trichromatic value, which can be used as a reference value for a precise trichromatic value.

Then, the reactive image can by analyzed by the mobile device to obtain a food safety information (step 206). FIG. 2B is a flowchart illustrating the detailed processes of the color correction process depicted in FIG. 2A according to an embodiment of the invention. The step for analyzing the reactive image by the mobile application of the mobile device may further comprise steps of: calculating the reactive image to generate a measured average value (step 208); generating a color correction function base on the reference value (step 210); and correcting the measured average value based on the color correction function to generate an exact result (step 212).

FIG. 2A shows that, after the address information is obtained according to the location of the mobile device (step 214), the food safety information and the address information are transmitted from the mobile device to a server through a wireless connection (step 216). When the server receives the food safety information and the address information, it maps the food safety information to a map (geographic map) according to the address information and publishes the map to the Internet (step 218).

Alternatively, the food safety testing sheet may include a lignocellulosic substrate. The reagent block (104) may also include a reagent (106) smeared on a lignocellulosic organization of the lignocellulosic substrate, wherein the specimen, which may be a liquid or a solution, is absorbed by the lignocellulosic substrate by capillary action when the lignocellulosic substrate contacts the specimen. The reagent then reacts with the specimen to detect the specimen when the specimen arrives at the reagent block (104).

The application for the camera in the invention, which is built on the basis of the Camera API of the camera function of the mobile device, can be customized and more flexible in design.

The camera function of the mobile application of the invention may inherit “Activity” and implement “SurfaceHolder.Callback” to receive information relevant to the lens preview interface. The camera function can perform three methods: (1) “surfaceCreated,” which can be called when the lens preview interface is built; (2) “surfaceChanged,” which can be called when the format or size of the lens preview interface are changed; and (3) “surfaceDestroyed,” which can be called when the lens preview interface is closed.

The processes of the invention comprise taking a picture, capturing images, analyzing the images, returning the results, and storing the results into the database. The detailed processes include (1) adding a “SurfaceView” to the layout file “manifest.xml” to enable preview; (2) adding “surfaceHolder” into “activity.java” to control “SurfaceView,” which varies with time; (3) building an application to trigger “surfaceCreated,” which is used for opening the lens of camera, setting relevant variables, and rendering images from lens of camera on “SurfaceView;” (4) using the “onClick” of “Button” in the auto focus function “Camera.autoFocus;” (5) using the auto focus monitor function “Camera.AutoFocusCallback” to detect the success of auto focus, and calling “Camera.PictureCallback” to take a picture; (6) calling “BitmapFactory.decodeByteArray” to transform data stream into Bitmap format; (7) obtaining the values for red, green, and blue colors of the Bitmap by using “Bitmap.getPixel when image recognition function detects Bitmap rectangular block (i.e., the reagent block);” (8) calculating the final value for the detection results when the relevant white balance algorithm is complete; (9) storing the resulting data into the database by network for various uses in the future.

Because the photograph quality is affected by the hardware of the mobile devices and environmental factors (such as light), color correction is required. Without a reference value, the mobile device cannot correct the RGB values of the reactive image. Therefore, the invention uses the trichromatic block printed on the food safety testing sheet to provide reference values for RGB colors and performs polynomial regression analysis to determine the true colors of the reactive image.

The three-dimensional n-order polynomial function can be obtained by polynomial regression analysis. For example, three-dimensional 2-order polynomial function is as follows:

R′=C(R,G,B)=a₀+a₁R+a₂G+a₃B+a₄R²+a₅RG+a₆G²+a₇RB+a₈GB+a₉B²

G′=C(R,G,B)=b₀+b₁R+b₂G+b₃B+b₄R²+b₅RG+b₆G²+b₇RB+b₈GB+b₉B²

B′=C(R,G,B)=c₀+c₁R+c₂G+c₃B+c₄R²+c₅RG+c₆G²+c₇RB+c₈GB+c₉B²

The R′, G′, and B′ are the average RGB values after the correction, and the R, G, and B are the measured RGB values before the correction. The R′, G′, and B′ can be a matrix of

$$[ \begin{matrix}{R}^{′} & {G}^{′} & {B}^{′}\end{matrix} ] = [ \begin{matrix}1 & R & G & B & {R}^{2} & RG & {G}^{2} & RB & GB & {B}^{2}\end{matrix} ] [ \begin{matrix}{a}_{0} & {b}_{0} & {c}_{0} \\ ⋮ & ⋱ & ⋮ \\ {a}_{9} & {b}_{9} & {c}_{9}\end{matrix} ]$$

The known exact value of trichromatic colors can be used to establish the matrix T, and the measured value of trichromatic colors can be used to create a matrix M. Finally, both values can be processed by performing polynomial regression analysis to obtain correction matrix C.

$$T = [ \begin{matrix}{R}^{′} & {G}^{′} & {B}^{′}\end{matrix} ]$$

$$M = [ \begin{matrix}1 & R & G & B & {R}^{2} & RG & {G}^{2} & RB & GB & {B}^{2}\end{matrix} ]$$

$$C = [ \begin{matrix}{a}_{0} & {b}_{0} & {c}_{0} \\ ⋮ & ⋱ & ⋮ \\ {a}_{9} & {b}_{9} & {c}_{9}\end{matrix} ]$$

$$T \approx MC$$

$$C = {( {M}^{T} M )}^{- 1} {M}^{T} T$$

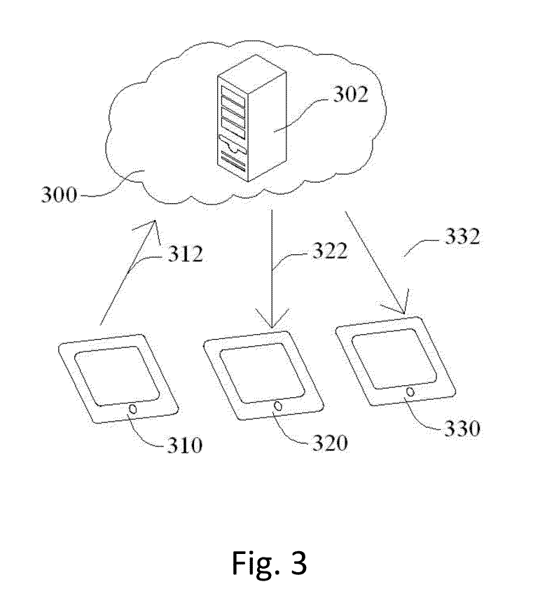

FIG. 3 is a flowchart of the process for creating a food safety map according another embodiment of the invention. For example, before a user dines at a restaurant, the testing results for nitrite concentrations can be obtained immediately by a mobile device (310). The testing results may be uploaded to the cloud (300) database (302) through a wireless network connection (312). Therefore, other users who visit the restaurant can use a mobile device with a wireless network connection (322) to check the food safety information of the restaurant published on the database (302) of the cloud (300).

Further, when many test results for a restaurant are sent to the cloud database, the test results are combined with the geographic map to construct a food safety map. The map can then be used to check the food safety information for a restaurant and to choose restaurants with good food safety records. Therefore, restaurants are motivated to improve their own food safety.

The foregoing description, for purposes of explanation, was set forth in specific details of the preferred embodiments to provide a thorough understanding of the invention. However, one skilled in the art would understand that specific details are not required in order to practice the invention. Therefore, the foregoing descriptions of specific embodiments of the invention are presented for purposes of illustration and description only and should not be construed in any way to limit the scope of the invention. The above description is not intended to be exhaustive or to limit the invention to the precise forms disclosed; many modifications and variations are clearly possible in view of the above teachings. The embodiments were chosen and described to provide the best explanation of the principles of the invention and its practical applications, thereby enabling others skilled in the art to best utilize the invention and various embodiments with various modifications as are suited to the particular use contemplated. It is intended that the following Claims and their equivalents define the scope of the invention.

The above embodiments of the invention are intended to be illustrative only. Numerous alternative embodiments may be devised by those skilled in the art without departing from the scope of the following claims.

What is claimed is:

1. A method for providing food safety map comprising steps of:
immersing a reagent block of a food safety testing sheet into a specimen, and waiting for a reactive period after the food safety testing sheet is taken out of the specimen wherein color of the reagent block changes after contacting the specimen;
using a camera of a mobile device to photograph the reagent block to obtain a reactive image of the reagent block that reacts with the specimen;
analyzing the reactive image by the mobile device to obtain a food safety information;
obtaining an address information according to location of the mobile device;
transmitting the food safety information and the address information to a server through a wireless connection by the mobile device;
using the server to receive the food safety information and the address information, and to map the food safety information to a map according to the address information; and
publishing the map to the Internet.

2. The method of claim 1, wherein the step of obtaining the reactive image of the reagent block further includes: photographing a trichromatic block mounted near the reagent block into a reference part of the reactive image.

3. The method of claim 2, wherein the step of analyzing the reactive image by the mobile device includes: calculating the reference part of the reactive image to generate a measured trichromatic value served as a reference value of an exactly trichromatic value.

4. The method of claim 3, wherein the step of analyzing the reactive image by the mobile device further comprises steps of:
calculating the reactive image to generate a measured average value;
generating a color correction function base on the reference value; and
correcting the measured average value based on the color correction function to generate an exact result.

5. The method of claim 1, wherein the food safety testing sheet includes a lignocellulosic substrate.

6. The method of claim 5, wherein the reagent block includes a reagent smeared on a lignocellulosic organization of the lignocellulosic substrate.

7. The method of claim 6, wherein the specimen, which is a liquid or a solution, is absorbed by the lignocellulosic substrate because of capillarity while the lignocellulosic substrate contacts the specimen, and the specimen reacts with the reagent when the specimen arrives the lignocellulosic organization.

8. A mobile application for providing food safety map comprising:
a photographing instruction for using a camera of a mobile device to photograph a reagent block of a food safety testing sheet to obtain a reactive image of the reagent block that reacts with a specimen after the reagent block contacts the specimen according to operation of a user, wherein color of the reagent block changes after contacting the specimen;
an analyzing instruction for analyzing the reactive image to obtain a food safety information;
a locating instruction for obtaining an address information according to location of the mobile device; and
a transmitting instruction for transmitting the food safety information and the address information to a server through a wireless connection by the mobile device,
wherein the food safety information and the address information are used by the server to map the food safety information to a map according to the address information.

9. The mobile application of claim 8, wherein the photographing instruction also photographs a trichromatic block mounted near the reagent block into a reference part of the reactive image.

10. The mobile application of claim 9, wherein the analyzing instruction further calculates the reference part of the reactive image to generate a measured trichromatic value served as a reference value of an exactly trichromatic value.

11. The mobile application of claim 10, the analyzing instruction further executes steps of:
calculating the reactive image to generate a measured average value;
generating a color correction function base on the reference value; and
correcting the measured average value based on the color correction function to generate an exact result.

12. A system for providing food safety map comprising:
a server for receiving and storing a food safety information from a mobile device of a user, mapping the food safety information to a map according to an address information, and publishing the map in the internet,
wherein the food safety information is generated from steps of: using a camera of a mobile device to photograph a reagent block of a food safety testing sheet to obtain a reactive image of the reagent block that reacts with a specimen after the reagent block contacts the specimen; and analyzing the reactive image, wherein color of the reagent block changes after contacting the specimen; and
the address information is obtained according to location of the mobile device when photographing the reactive image.

13. The system of claim 12, wherein the reactive image is analyzed by a mobile application of the mobile device.

14. The system of claim 12, wherein the food safety testing sheet includes a lignocellulosic substrate.

15. The system of claim 14, wherein the reagent block includes a reagent smeared on a lignocellulosic organization of the lignocellulosic substrate.

16. The system of claim 15, wherein the specimen, which is a liquid or a solution, is absorbed by the lignocellulosic substrate because of capillarity while the lignocellulosic substrate contacts the specimen, and the specimen reacts with the reagent when the specimen arrives the lignocellulosic organization.
